(19) United States
(12) Patent Application Publication
(10) Pub. No.: US 2008/0234601 A1
(43) Pub. Date: Sep. 25, 2008

(54) VISUAL STIMULATION METHOD USING LEVELS OF EYE CONTACT FOR DIAGNOSIS AND TREATMENT OF MENTAL DISORDERS

(76) Inventor: David Wexelman, Brooklyn, NY (US)

Correspondence Address: DONALD W. MEEKER, 924 EAST OCEAN FRONT, # E, NEWPORT BEACH, CA, 92661, US

(21) Appl. No.: 12/048,367

(22) Filed: Mar. 14, 2008

(30) Foreign Application Priority Data
Mar. 25, 2007 (IL) 182158

(51) Int. Cl. A61B 13/00

(52) U.S. Cl. 600/558

(57) ABSTRACT

A method for mental health diagnosis and treatment using visual stimulation techniques. Eye to eye contact, face to face contact, looking up, looking to the side, back turned, finger movement in front of the eyes, and viewing multiple images of the patient are employed in the method. The method is used with a therapist or by a patient using a self help kit. Computer and Web camera techniques are used with programs for use with a therapist and a self help program.

### CROSS-REFERENCE TO RELATED APPLICATIONS

The present US utility patent application claims the benefit of Israeli patent application #182,158 filed Mar. 25, 2007.

### STATEMENT REGARDING FEDERALLY SPONSORED RESEARCH OR DEVELOPMENT

Not Applicable.

### THE NAMES OF THE PARTIES TO A JOINT RESEARCH OR DEVELOPMENT

Not Applicable.

### BACKGROUND OF THE INVENTION

### Field of the Invention

The present invention relates to Description of Related Art including information disclosed under 37 CFR 1.97 and 1.98

Many people have become mentally disabled because their problem was not detected in its early stages and treatment was not given to them on time. Many soldiers chosen for combat have become mentally disabled because their minor mental problem was not detected at the time of enlistment into the military. Many people are misdiagnosed to have mental problems when their problems are physical. They are hospitalized and treated improperly which results in serious complications mentally and physically.

People with mental disorders such as anxiety and depression are super sensitive to eye contact and to their own image as it is reflected on the screen of the computer by a Web camera or Webcam. A therapist can often notice the sensitivity of their patients to making eye contact. Sometimes it is not obvious. They are also sensitive to various questions which the therapist may ask the patient. Sensitivity to eye contact is a general indication that there is an underlying psychological problem. Specific problems are diagnosed and treated by the therapist through asking questions.

Psychologists have discovered that through changing eye movements stress can be released. The technique of using eye movements in therapy especially trauma therapy is called Eye Movement Desensitization and Reprocessing (EMDR). It is accepted today in psychology that understanding the connection between the eye and the mind can open up channels for helping people with mental illness.

The inventor of the present invention, who is also the inventor of Visual Stimulation Devices in U.S. Pat. No. 5,388,994, has had the opportunity to explore, understand and apply Visual Stimulation in simulation of eye contact both in high tech and as a healer.

The inventor of the present invention has made a number of discoveries. The nervous system and the mind are interwoven. The mind receives neurological messages which keep it calm. The nervous system works 24 hours a day while the mind rests and sleeps. The rhythm of the respiratory system sends psycho respiratory messages to the mind to calm the mind. Psycho neurological messages are light. Psycho respiratory messages are sound. Trauma causes a Psycho neurological blockage. The mind does not receive these messages like a healthy person. Trauma can also aggravate the rhythm of the respiratory system. The eye which is connected to light is connected to the nervous system and these neurological messages. The ear is connected to the sound of the breath. With the eye on its highest level of Visual Stimulation, psycho neurological blockages which cause anxiety and depression can be diagnosed. In diagnosis a neurological message is simulated through which a blockage can be detected.

While the prior art provides some early detection and early intervention techniques, none of them provide an intensive self-help program using a personal computer (PC) with a Webcam for visual therapy.

In U.S. Pat. No. 5,388,994 for a Visual Stimulation Devices issued to the present inventor, mirrors are used which revolve at high speed and slow hypnotic speeds. It utilizes momentary serial reflections of a user from a mirror with interspersed exposure to opaque colored areas, pictures, letters, numbers etc. The repetition of exposure is variable under the control of the user and above certain cyclic rates, the impression upon the user is totally subliminal and at the slowest speeds, completely hypnotic. Computer technology has enabled expansion of the method. The visual effects of this patent can now be duplicated and improved by Personal Computers (PCs) for diagnosis and treatment of mental disorders. This patent was issued at the time Windows was developing. Research using the present inventor's prior patent, which is still active in Israel, has paved the way for the present invention including diagnostic techniques and healing techniques which were not in the prior patent. The present invention joins the more recent technology and development using computer technology to make the PC an excellent tool for therapy.

The following are some of the prior art patents that have been written using computer technology in diagnosis and treatment of mental disorders since the issuing of the inventor's prior U.S. Pat. No. 5,388,994 incorporated herein by reference.

U.S. Pat. No. 7,115,099, issued Oct. 3, 2006 to Miller, provides a method and apparatus for diagnosis of a mood disorder.

U.S. Pat. No. 6,572,562, issued Jun. 5, 2003 to Marshall, discloses methods for monitoring affective brain functions. This patent of a method called Eyetracking reveals the affects of visual stimulus on the pupils positive and negative.

U.S. Pat. No. 6,530,884, issued Mar. 11, 2003 to Balkin, is for a method and system for predicting human cognitive performance.

U.S. Pat. No. 6,520,905 provides a Survey Management of the physiological and psychological state of an individual using images portable biosensor device. This patent shows that the use of specific images affect the mind in specific ways.

U.S. Pat. No. 6,334,778 Jan. 1, 2002 issued to Brown, shows a remote psychological diagnosis and monitoring system.

U.S. Pat. No. 6,306,077, issued Oct. 23, 2001 to Prabhu Hurwitz, provides management of the physiological and psychological state of an individual using images as an overall system. This patent uses selected images for management of the psychological state of an individual.

U.S. Pat. No. 6,309,342, issued Oct. 30, 2001 to Blazey, puts forth the management of the physiological and psychological state of an individual using images and a biometrical analyzer. The biometric analyzer measures the effects of selected visual images on the patient.

U.S. Pat. No. 6,293,904 issued to Blazey, is for management of the physiological and psychological state of an individual using personal image profiler. In this patent various images are used to manage the psychological state and the effects are measured by a biometric analyzer and personal image profiler.

U.S. Pat. No. 6,057,846, issued Mar. 2, 2000 to Sever, provides a virtual reality psychophysiological conditioning medium, In this patent subliminal stimulus is applied for psycho-physiological conditioning.

U.S. Pat. No. 6,012,926, issued Jan. 11, 2000 to Hodges, uses virtual reality for treating patients with anxiety disorders. A virtual Reality environment is monitored by diagnostic equipment in image therapy for treatment of phobias.

U.S. Pat. No. 5,742,748, issued Apr. 21, 1998 to Sever, provides a virtual reality mental conditioning medium. This is one of the first patents applying computer technology in creating virtual reality for mind conditioning.

In many of the above patents visual stimulus has been applied and monitored. The effects of visual stimulus on the mind has already been monitored and applied in treatment. The visual stimulus of the present invention produced by a Webcam and its application in diagnosis and treatment has not been disclosed in the prior art patents. The concept of simulation of eye contact on a computer has not been discussed.

### BRIEF SUMMARY OF THE INVENTION

In the Visual Stimulation method, there are six levels of eye contact and eye movement:

1. Looking straight into another person's eyes;

2. Looking straight at another person face to face as when one person greets the other, they shake hands and smile at each other;

3. Looking straight at the face of another person when the other person is looking up or the visual effect of looking up;

4. Looking at another person when the other person face is turned to the side;

5. Looking at another person when his back is turned;

6. Following a persons finger as it moves slowly in front of your eyes, called EMDR.

All these six levels of eye contact can be produced by a therapist with a patient and by a patient in self help sessions using a kit comprising physical items and computer programs used with a personal computer and Web camera (Webcam) with the Visual Stimulus steps simulated on a personal computer through using the Webcam and applied in diagnosis and healing mental disorders. The disclosure of the present invention explains how to produce each level of eye contact on the computer, and the visual effect of EMDR. It explains how to apply these visual effects in diagnosis and treatment of anxiety and depression.

In brief, the personal computer has the potential today to be an excellent tool in therapy. Today every computer comes with a drive for using a Webcam. The disclosure of the present invention relates to techniques of using visual effects created by the Webcam simulating levels of eye contact applied in diagnosis and treatment of mental disorders. The work of psychologists and psychiatrists sometimes demands emotional involvement which limits their ability to help their patients, especially those patients who suffer from psychosis. The present invention adds to the computer the very necessary visual connections that exist between the therapist and his patients in such a way to allow the computer to assist the doctor in saving lives.

All people who have even minor depression, anxiety, and mental disorders are supersensitive to the stimulus of the present invention, which is simulation of intensive eye contact using the unique application of the Web camera. This makes this stimulus ideal for use in diagnosis. Only the visual stimulus of the present invention can detect the signs of mental illness in its early stages to enable early intervention and treatment.

The Level #1 Visual Stimulus program of the present invention is the highest most intense level and most suitable for detecting the smallest mental problem in a general context without knowing its causes. The present invention also provides a technique of treatment, simulated on a computer, of the visual effects of the eye movements in the treatment EMDR which is used in trauma therapy. This will allow EMDR to be applied in computer programs, one designed for use with a professional and one designed for self help.

Mental illness in early stages can be detected and treated using the techniques of the present invention.

The technique of diagnosis of the present invention can be valuable to the military in choosing which soldiers are suitable for combat.

### BRIEF DESCRIPTION OF THE SEVERAL VIEWS OF THE DRAWINGS

These and other details of the present invention will be described in connection with the accompanying drawings, which are furnished only by way of illustration and not in limitation of the invention, and in which drawings:

FIG. 1 is an elevational view of a patient working with a mental health professional to carry out step 1 of the Visual Stimulation method using an opaque cup initially covering the patient's eyes and then lowering the cup to enable looking straight into another person's eyes;

FIG. 2 is an elevational view of a patient working with a mental health professional to carry out step 2 of the Visual Stimulation method using an opaque cup on a table in front of a patient with the patient first looking down at the cup and then looking straight at another person face to face as when one person greets the other, they shake hands and smile at each other;

FIG. 3 is an elevational view of a patient working with a mental health professional to carry out step 3 of the Visual Stimulation method using an opaque cup lifted up in front with the patient looking up at the cup to produce the effect of looking straight at the face of another person when the other person is looking up or the visual effect of looking up;

FIG. 4 is an elevational view of a patient working with a mental health professional to carry out step 4 of the Visual Stimulation method with the patient looking off to one side to produce the visual effect of looking at another person when the other person face is turned to the side;

FIG. 5 is an elevational view of a patient working with a mental health professional to carry out step 5 of the Visual Stimulation method with the patient's back turned to produce the effect of looking at another person when his back is turned;

FIG. 6 is an elevational view of a patient working with a mental health professional to carry out step 6 of the Visual Stimulation method with the patient following a persons finger as it moves slowly in front of the patient's eyes, called EMDR;

FIG. 7 is an elevational view of a patient working with a mental health professional or alone using mirrored glasses to look into a mirror so that the patient can see multiple images of himself in the mirror to produce the effects of visual stimulation devices;

FIG. 8 is an elevational view of a patient working with a mental health professional with the use of program 1 or working alone with the use of program 2, in both cases, using a personal computer with a Webcam so that the user can view the user's own face on the computer screen produced by the Webcam to produce the results of step 1 of the Visual Stimulation method which may involve using an opaque cup initially covering the patient's eyes and then lowering the cup to enable looking straight into another person's eyes;

FIG. 9 is an elevational view of a patient working with a mental health professional with the use of program 1 or working alone with the use of program 2, in both cases, using a personal computer with a Webcam so that the user can view the user's own face on the computer screen produced by the Webcam to produce the results of step 1 of the Visual Stimulation method which may involve using an opaque cup initially covering the patient's eyes and then lowering the cup to enable looking straight into another person's eyes, showing the back of the patient looking into the computer screen;

FIG. 10 is an elevational view of a patient working with a mental health professional with the use of program 1 or working alone with the use of program 2, in both cases, using a personal computer with a Webcam so that the user can view the user's own face on the computer screen produced by the Webcam to produce the results of step 2 of the Visual Stimulation method which may use an opaque cup on a table in front of a patient with the patient first looking down at the cup and then looking straight at another person face to face as when one person greets the other, they shake hands and smile at each other;

FIG. 11 is an elevational view of a patient working with a mental health professional with the use of program 1 or working alone with the use of program 2, in both cases, using a personal computer with a Webcam so that the user can view the user's own face on the computer screen produced by the Webcam to produce the results of step 2 of the Visual Stimulation method which may use an opaque cup on a table in front of a patient with the patient first looking down at the cup and then looking straight at another person face to face as when one person greets the other, they shake hands and smile at each other, showing the back of the patient looking into the computer screen;

FIG. 12 is an elevational view of a patient working with a mental health professional with the use of program 1 or working alone with the use of program 2, in both cases, using a personal computer with a Webcam so that the user can view the user's own face on the computer screen produced by the Webcam to produce the results of step 3 of the Visual Stimulation method using an opaque cup lifted up in front with the patient looking up at the cup to produce the effect of looking straight at the face of another person when the other person is looking up or the visual effect of looking up;

FIG. 13 is an elevational view of a patient working with a mental health professional with the use of program 1 or working alone with the use of program 2, in both cases, using a personal computer with a Webcam so that the user can view the user's own face on the computer screen produced by the Webcam to produce the results of step 3 of the Visual Stimulation method using an opaque cup lifted up in front with the patient looking up at the cup to produce the effect of looking straight at the face of another person when the other person is looking up or the visual effect of looking up, showing the back of the patient looking into the computer screen;

FIG. 14 is an elevational view of a patient working with a mental health professional with the use of program 1 or working alone with the use of program 2, in both cases, using a personal computer with a Webcam so that the user can view the user's own face on the computer screen produced by the Webcam to produce the results of step 4 of the Visual Stimulation method with the patient looking off to one side to produce the visual effect of looking at another person when the other person face is turned to the side;

FIG. 15 is an elevational view of a patient working with a mental health professional with the use of program 1 or working alone with the use of program 2, in both cases, using a personal computer with a Webcam so that the user can view the user's own face on the computer screen produced by the Webcam to produce the results of step 4 of the Visual Stimulation method with the patient looking off to one side to produce the visual effect of looking at another person when the other person face is turned to the side, showing the back of the patient looking into the computer screen;

FIG. 16 is an elevational view of a patient working with a mental health professional with the use of program 1 or working alone with the use of program 2, in both cases, using a personal computer with a Webcam so that the user can view the user's own face on the computer screen produced by the Webcam to produce the results of step 5 of the Visual Stimulation method with the patient's back turned to produce the effect of looking at another person when his back is turned;

FIG. 17 is an elevational view of a patient working with a mental health professional with the use of program 1 or working alone with the use of program 2, in both cases, using a personal computer with a Webcam so that the user can view the user's own face on the computer screen produced by the Webcam to produce the results of step 5 of the Visual Stimulation method with the patient's back turned to produce the effect of looking at another person when his back is turned, showing the back of the patient looking into the computer screen;

FIG. 18 is an elevational view of a patient working with a mental health professional with the use of program 1 or working alone with the use of program 2, in both cases, using a personal computer with a Webcam so that the user can view the user's own face on the computer screen produced by the Webcam to produce the results of step 6 of the Visual Stimulation method with the patient following a persons finger as it moves slowly in front of the patient's eyes, called EMDR;

FIG. 19 is an elevational view of a patient working with a mental health professional with the use of program 1 or working alone with the use of program 2, in both cases, using a personal computer with a Webcam so that the user can view the user's own face on the computer screen produced by the Webcam to produce the results of step 6 of the Visual Stimulation method with the patient following a persons finger as it moves slowly in front of the patient's eyes, called EMDR, showing the back of the patient looking into the computer screen;

FIG. 20 is an elevational view of a patient working with a mental health professional with program 1 or alone using program 2, both with a personal computer and a Webcam producing a series of images of the patient on the computer screen to produce the effect of the mirrored glasses of FIG. 7 to look into a mirror so that the patient can see multiple images of himself in the mirror to produce the effects of visual stimulation devices;

FIG. 21 is a perspective view showing the components of the Visual Stimulation kit comprising an opaque cup, mirrored glasses, program 1 on a DVD disc to be used with a mental health professional, and program 2 on a DVD disc to be used by a patient for self help, and also showing (in dashed lines) a personal computer and a Web camera to be used with the two programs.

### DETAILED DESCRIPTION OF THE INVENTION

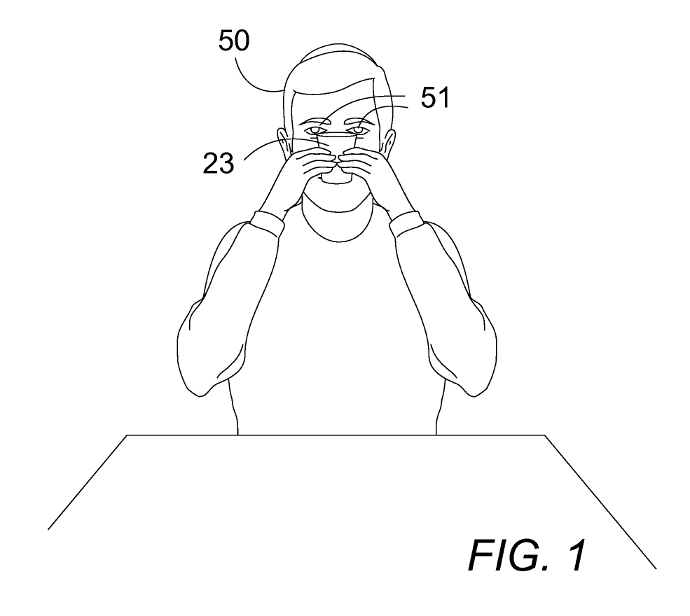

In FIGS. 1-21, a Visual Stimulus method of analysis uses Eye Contact and Eye Movements for diagnosis and treatment of psychological problems.

In FIGS. 1-6, the Visual Stimulus method comprises carrying out a six step Visual Stimulus method of analysis using Eye Contact and Eye Movements for diagnosis and treatment of psychological problems, comprising the six steps of:

1. Looking straight into the eyes of a person, as shown in FIG. 1 using a cup 23 to cover and then reveal the eyes 51 of the person 50;

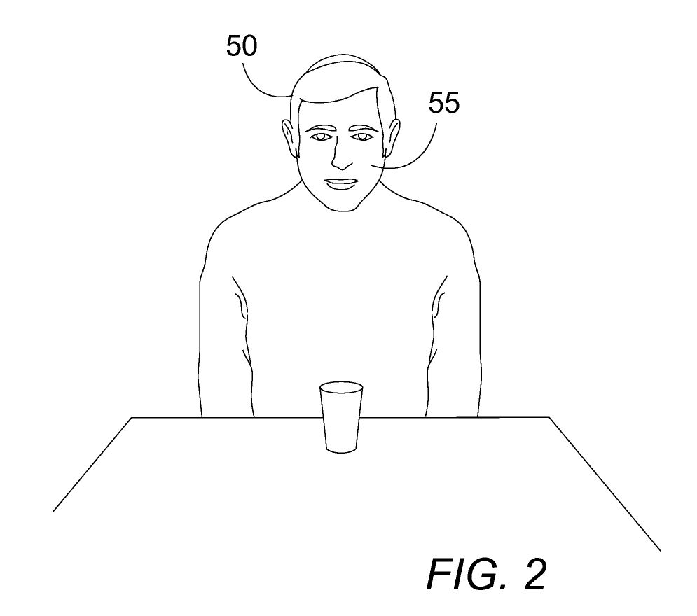

2. Looking straight at the person face 55 to face as when one person 50 greets the other and they shake hands and smile at each other, as shown in FIG. 2;

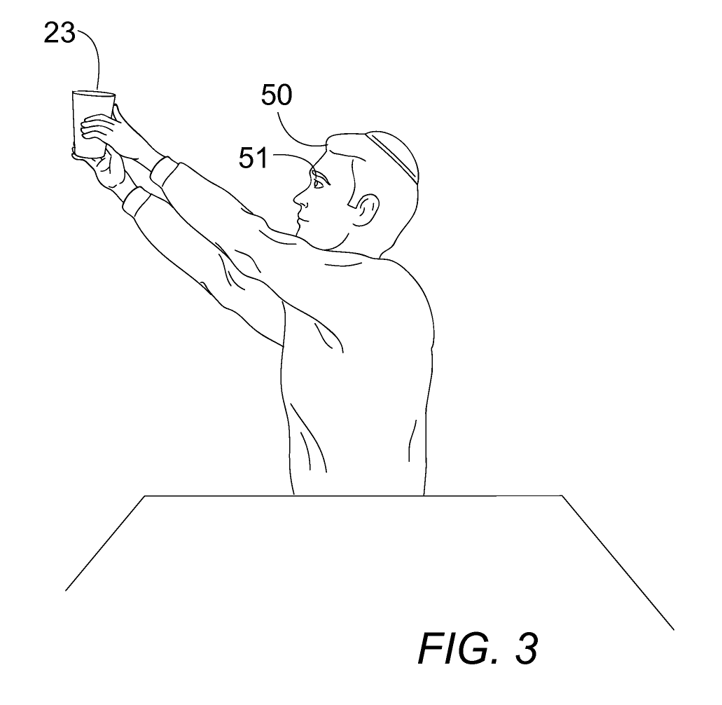

3. Looking straight at the face 55 of the person when the person 50 is looking up at the cup 23 held aloft, as shown in FIG. 3;

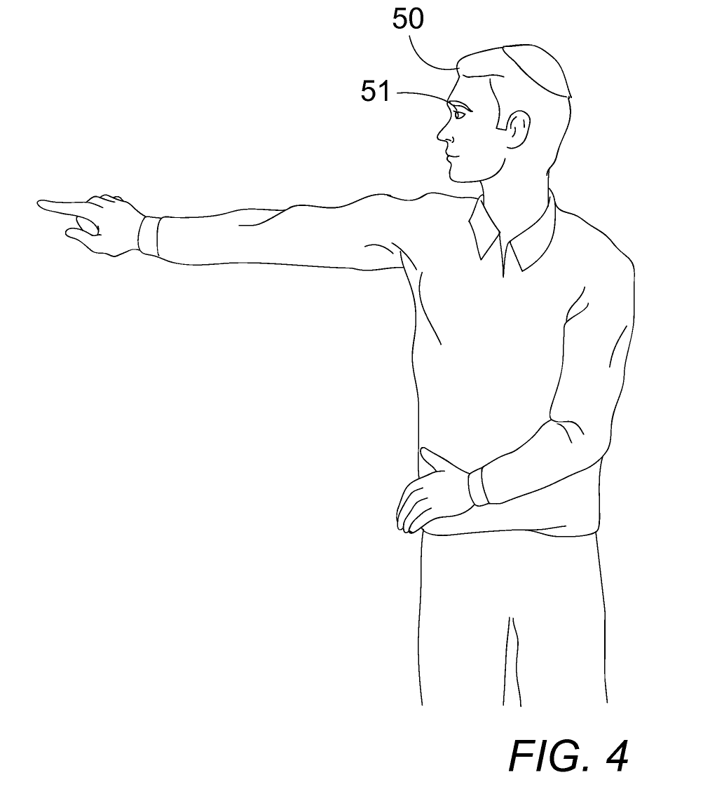

4. Looking at the face 55 of the person 50 when the person's face is turned to the side, as shown in FIG. 4;

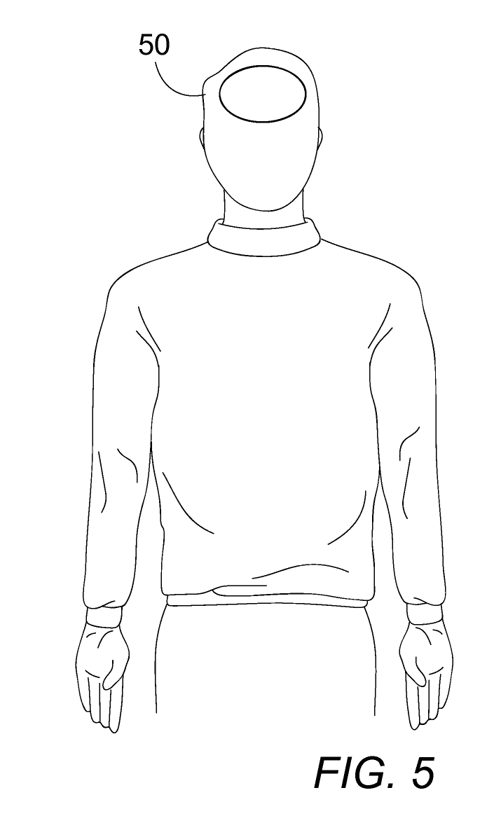

5. Looking at the person 50 when the person's back is turned, as shown in FIG. 5;

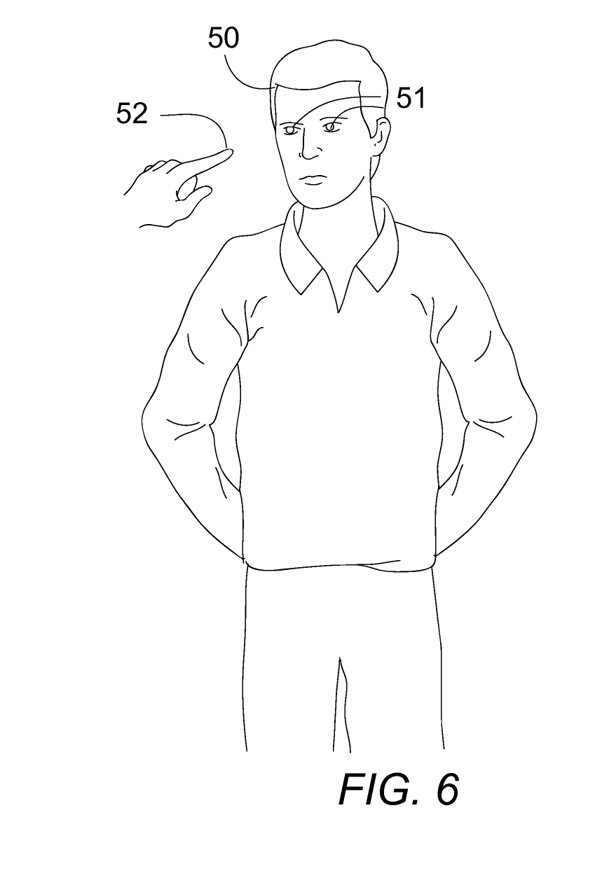

Following the person's finger 52 as it moves slowly in front of the eyes 51 in a procedure called EMDR, as shown in FIG. 6.

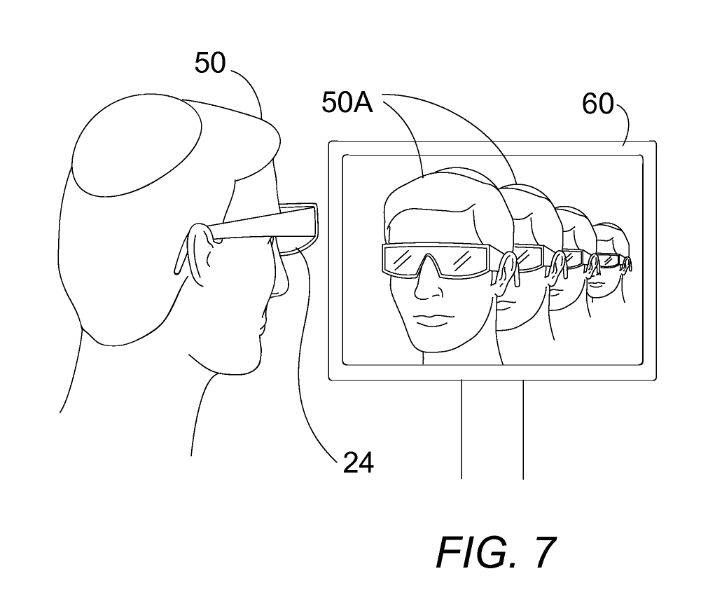

In FIG. 7, a person 50 uses mirrored glasses 24 to look into a mirror 60 so that the person can see multiple images of himself in the mirror to produce the effects of visual stimulation devices.

In one embodiment, a therapist employs the six steps of the method with a patient.

In an alternate embodiment, a person employs the six steps of the method while looking into a mirror.

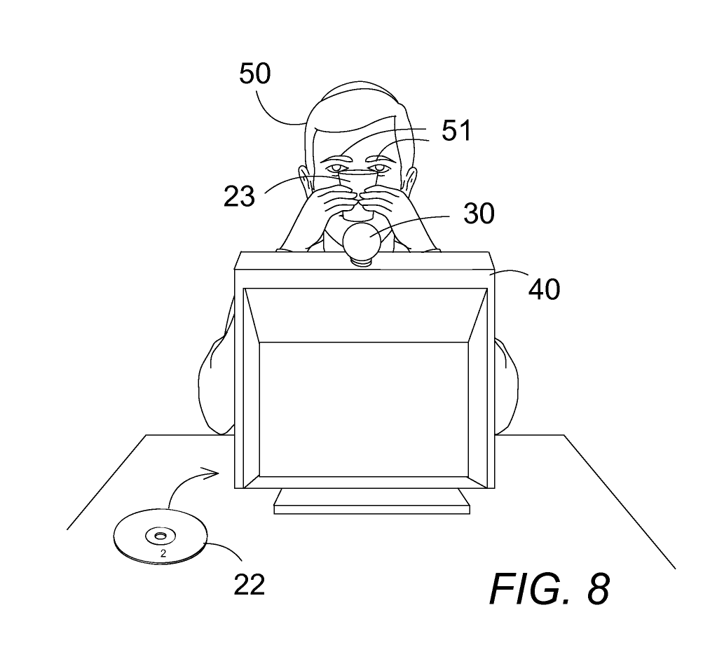

In a third embodiment, a person employs the six steps of the method by looking into a computer screen having the person's image on the screen from a video camera pointed at the person. As shown in FIGS. 8-20. In this embodiment, the system uses a software program played on a computer wherein the software program carries out the therapeutic steps of the six step method for the person using the computer.

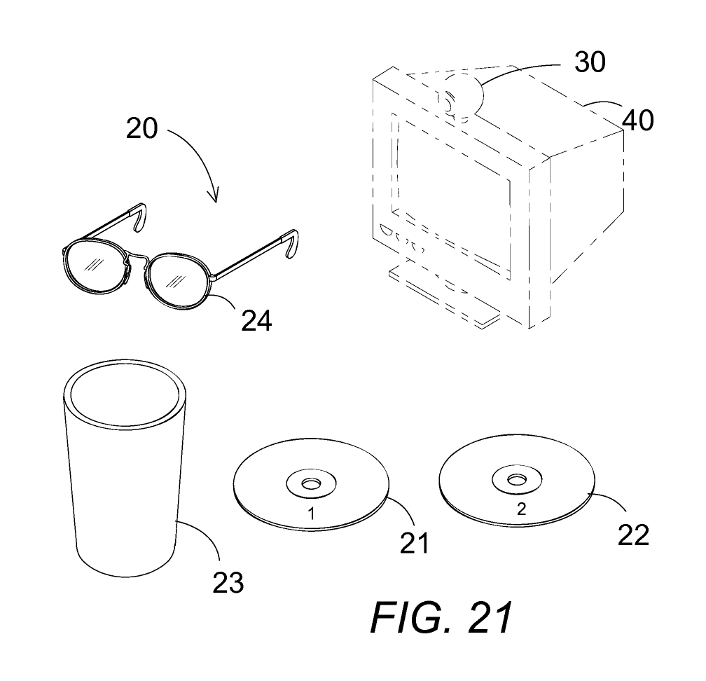

A kit is provided to assist in performing the six steps of the method, as shown in FIG. 21. The kit contains:

a disc 21 containing a software program 1, to be administered by a professional therapist, for carrying out the steps providing the therapeutic effects of the six step method for a person using a computer;

a disc 22 containing a software program 2, to be used on a computer by a person without the presence of a professional therapist, for carrying out the steps providing the therapeutic effects of the six step method;

a cup 23, to be used as a focusing device by a person using the six step method;

a pair of mirrored glasses 24, for creating multiple images of a person looking into a mirror, while carrying out the method.

In FIGS. 8-20, a Visual Stimulus method of analysis uses a personal computer 40 and a Web camera 30 simulating Eye Contact and Eye Movements for diagnosis and treatment of psychological problems. The method comprises:

a user 50 looking into a computer screen 41 while a Web camera 30 is projecting a real time image of the user on the computer screen;

the user carrying out the six steps of the Visual Stimulus method of analysis simulating Eye Contact and Eye Movements for diagnosis and treatment of psychological problems, comprising the six steps of:

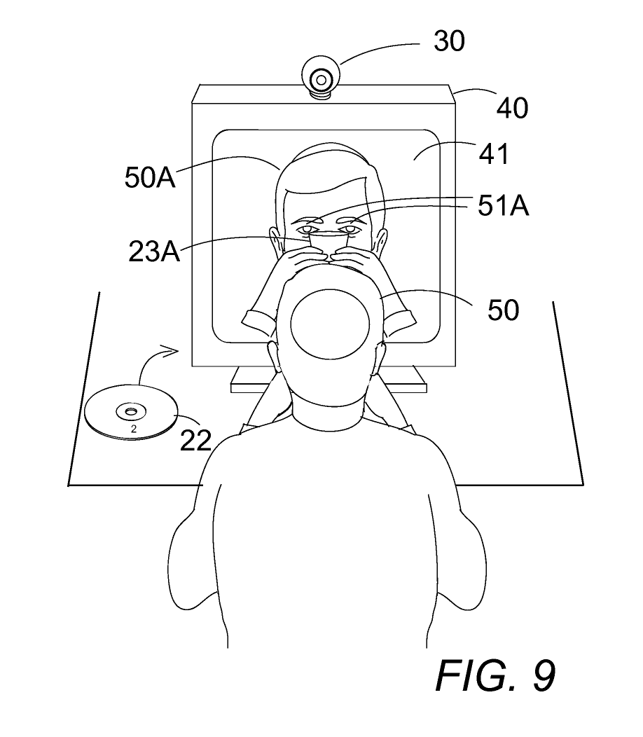

1. the user 50 looking straight into the user's own eyes on a computer screen 41 using a cup 23, 23A to cover and then reveal the eyes 51, 51A of the person 50, as shown in FIGS. 8 and 9;

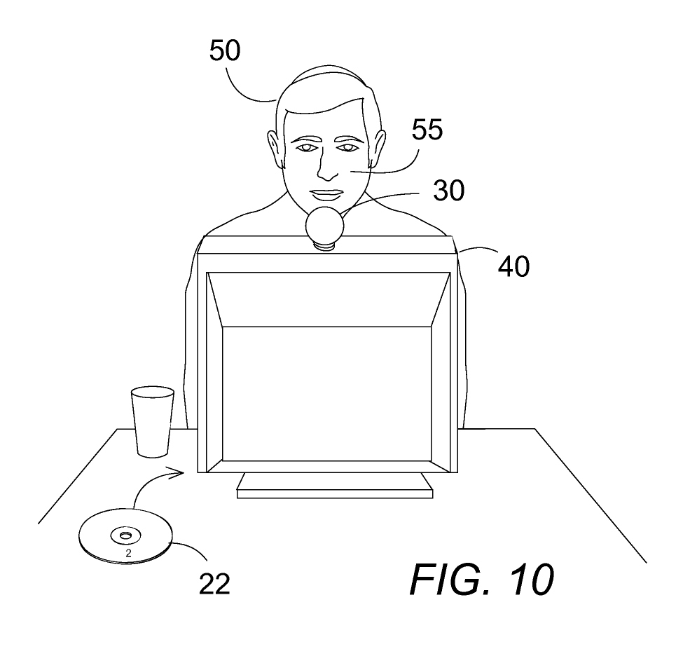

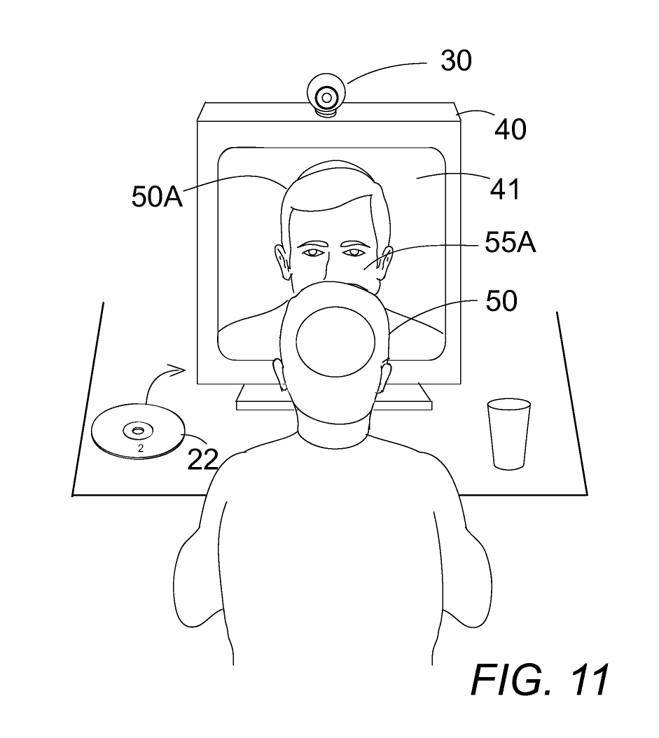

2. the user 50 looking straight at the user's image on the computer screen 41 face to face 55, 55A as when one person greets the other, and they shake hands and smile at each other, as shown in FIGS. 10 and 11;

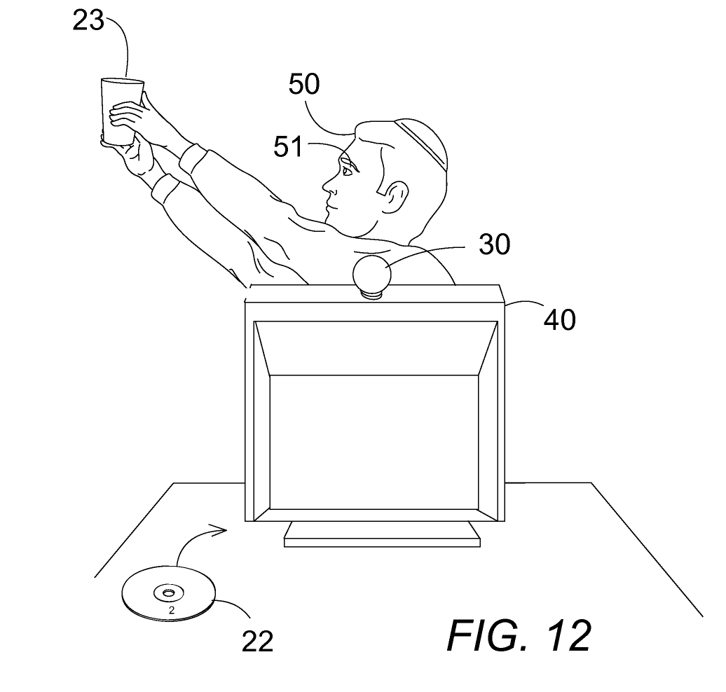

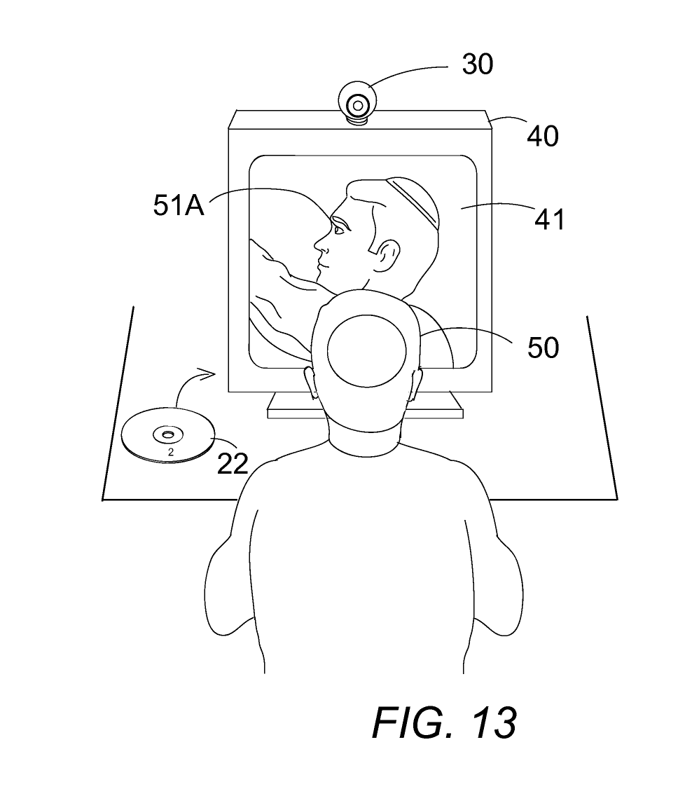

3. the user 50 looking at the user's own face 55A when the user's face is looking up on the computer screen 41 or the visual effect of looking up, as shown in FIGS. 12 and 13;

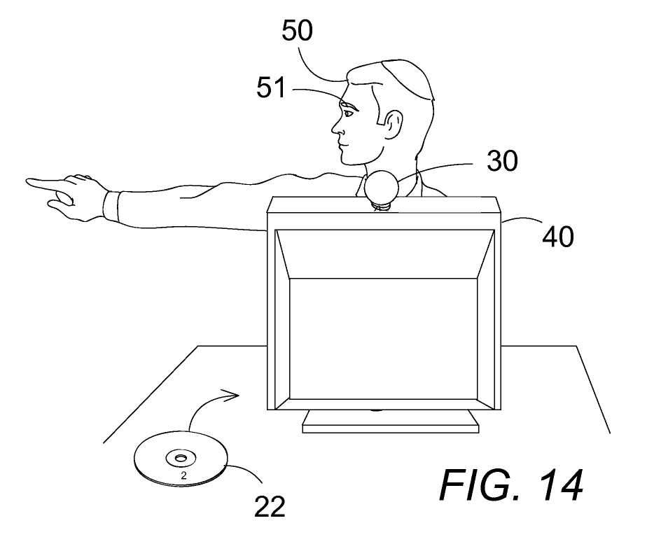

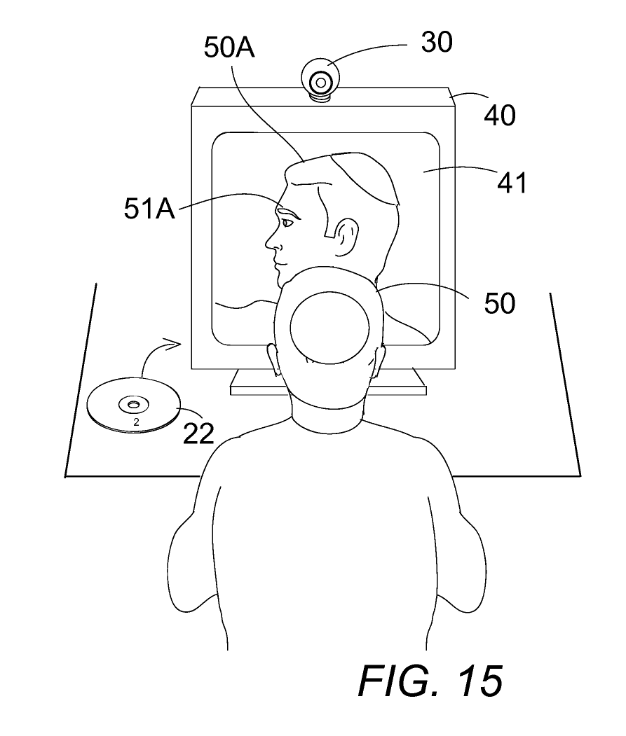

4. the user 50 looking at the user's own face 55 when the user's face 55A is turned to the side in the computer screen 41, as shown in FIGS. 14 and 15;

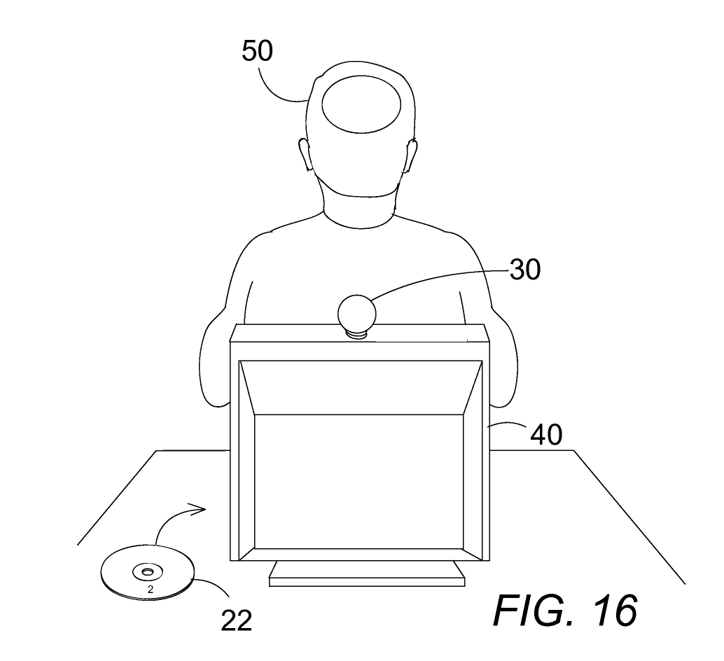

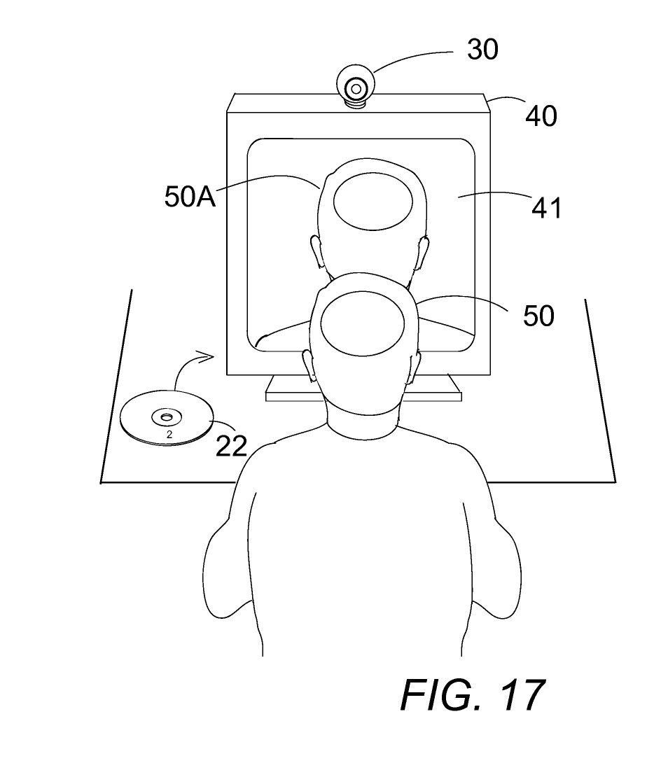

5. the user 50 looking at the user's own image 50A when the user's back is turned in the computer screen 41, as shown in FIGS. 16 and 17;

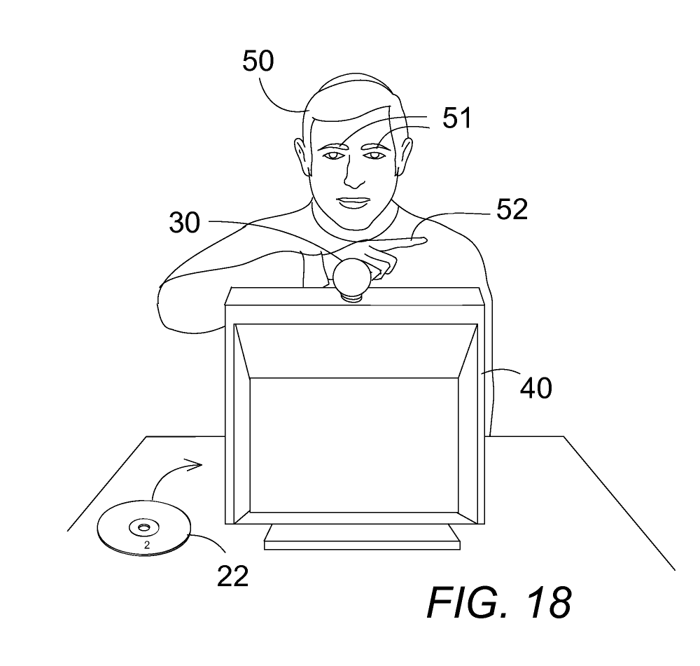

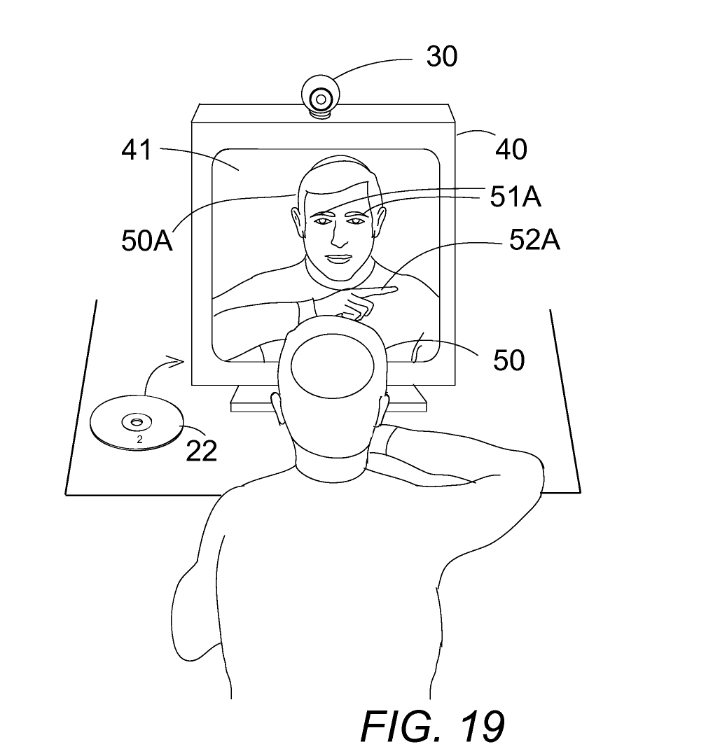

6. the user 50, 50A following a finger 52, 52A on the computer screen 41 as it moves slowly in front of the user's eyes 51, 51A, as shown in FIGS. 18 and 19, in a procedure called EMDR.

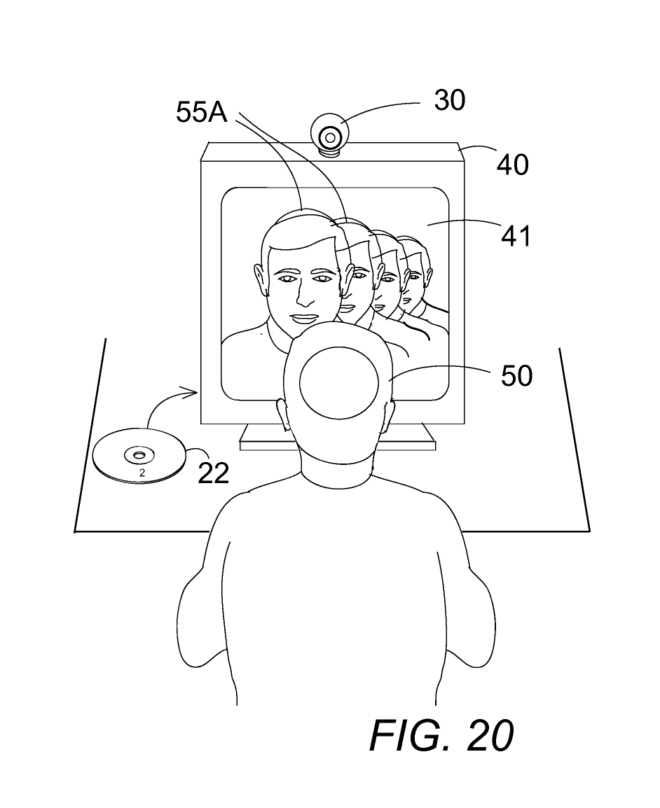

In FIG. 20 a person works with a mental health professional with program 1 or alone using program 2, both with a personal computer and a Webcam producing a series of images of the patient on the computer screen, producing the effect of the mirrored glasses of FIG. 7 to produce the effects of visual stimulation devices.

In use, the Visual Stimulus method of analysis uses Eye Contact and Eye Movements for diagnosis and treatment of Mental Disorders in their early stages and for recruitment of soldiers for combat, using the specific Visual Stimulus described in the six-step method, as well as EMDR technique, both in person with a professional therapist, and through simulation of Eye Contact using a Web camera and a computer program on a PC.

There are six levels of eye contact and eye movement and they may be derived and reproduced on a computer screen using the Web camera.

Level #1: Looking straight into another person's eyes. There is a difference between looking at a person face to face and looking directly into his eyes. There is a technique in therapy for checking eye contact using a cup as an intermediate. First you have the person look at the cup. You raise up the cup above the person's eye level. You slowly lower the cup and then you make eye contact. You isolate the eyes from the other parts of the face in this way. This is what is meant by “eye contact looking straight into another person's eyes.” On this level the eye stress is much higher than face to face. This level of eye contact is simulated and programmed onto the computer in this way. With the Webcam focused on the person, the person is instructed by the computer to look at the screen. He will see himself on the screen. Then the Webcam flashes the same live image 150 times a minute onto the screen. There is both the image of the person visible to his eye on the screen and the flashing of his image which is subliminal at these high speeds.

Level #2: Face to face. It is very well known that people very often feel better after going to a doctor even before they take his medicines. The initial contact between two people when they greet each other has a great effect upon the rest of their meeting. There is healing energy in face to face eye contact. When you shake hands or hug more energy is added. Face to face eye contact is simulated and programmed into the computer in this way: a still picture first appears on the screen. The picture can be of the therapist, a beautiful landscape scene, an American flag for those that love America, etc. The live image of the person is flashed onto the screen at subliminal speeds of 150 times a minute, superimposed onto the initial still picture. This level contains the same flashing as in Level #1 but is lower in intensity than Level #1 because there is a still picture and not the live image of the person himself.

Level #3: Looking straight at the face of another person when the other person is looking up, or the visual effect of looking up. In this level, the person is not looking at the other person at all. He is looking upward. There is no direct eye contact. When there is direct eye contact there is flashing with the Webcam. Looking upward has a hypnotic effect. People look upward when they pray. Looking upward has a natural healing effect. This effect is hinted about in the Book of Psalms chapter 122, “Lift your eyes to the mountain, from where will come your help.” Eye movement of looking upward is used in therapy for stress release.

The visual effect of looking upward simulated and programmed into the computer is a slow hypnotic chain of pictures in conjunction with the image of the user focused on the screen with the Webcam. A system of seven pictures has already been established in U.S. Pat. No. 5,388,994. These pictures should be related. For example six animals and the live image of the person as the seventh interval, or six beautiful landscapes and the live image of the person as the seventh interval. The pictures appear on the screen slowly, one at time, at one minute intervals. This flow is repeated for three revolutions of pictures flowing at a hypnotic speed.

Level #4: Looking at another person when the other person's face is turned to the side. In Level #4, since the person is not facing the other person, the visual stimulation is on a much lower level than Level #2. Most teachers and psychologists are not looking directly at their students or patients. There is indirect visual eye contact, which occurs at Level#3. There is occasional visual stimulation, depending upon the degree the therapist is turned to the side. The larger the degree, the less visual stimulation. Level #3 is simulated on the computer screen like Level #2. There is a still picture on the screen but the flashing with the Webcam is much slower depending upon the degree to which the face is turned to the side.

When the face is already turned to the side completely and there is no face to face contact, there is then no flashing at all, but there is a similarity to looking at a seven interval sequence with the live image on the seventh interval. Whereas in looking up simulation, the seven intervals revolved at hypnotic speed of one interval per minute, in the looking to the side simulation, the interval is seven revolutions per minute. This effect can help in stress release.

Level #5: Looking at another person when his back is turned. Level #5 is like looking into a mirror. You see yourself on the computer screen. There is no flashing of the image and no movement of the image on the screen.

Level #6: Following a person's finger as it moves slowly in front of your eyes (EMDR). This level of Visual Stimulations is simulated like a revolving prism at slow speeds. There are three visual stimuli which revolve slowly, one of which is the live image created with the webcam. They begin to revolve at three revolutions per minute and slow down to two revolutions per minute. This produces a hypnotic effect which performs like EMDR eye movement therapy for trauma. This effect can be used in self help computer software programs similarly to the way it is applied in therapy.

The eye has in it a reflective hypnotic power and the sense of sight. The mind is connected to the eye. You see and you think. When you look at the computer where a Webcam is mounted on the computer pointing in your direction you are also looking at yourself. You can see yourself on the screen if the Webcam is connected. This is called a two way visual connection between the computer and its user. In this way the Webcam acts like a mirror.

U.S. Pat. No. 5,388,994 disclosed two embodiments with mirrors planted on them: a mirror mounted on a prism which revolved at over 100 revolutions per minute, and a mirror mounted on a wheel that revolved at slow speeds of one revolution per minute. Both embodiments had hypnotic visual effects. Computer programming can create these effects as well as other visual effects with the Webcam, specifically the simulation of the six levels of eye contact describer above. These visual effects can be incorporated and programmed with other innovations in computer-based therapy, making the computer an excellent tool for therapy, almost as effective as a live professional psychologist. The computer with the Webcam can test sensitivity to eye contact when it is programmed to create and simulate the level of eye contact created by looking straight into another person's eyes. With diagnostic equipment the sensitivity of the patient to the eye contact can be detected with the Webcam and computer.

By changing the level of eye contact from Level #1 to Level #3 to Level #2 and back to Level #3, the sensitivity of the patient to eye contact can be treated. The process is continued beginning with Level #1 until the diagnostic equipment indicates that the stress and sensitivity to this eye contact created on Level #1 has been released.

These are the steps in diagnosis and treatment of mental disorders applying the Webcam on the computer.

### Diagnosis of Mental Disorders Using Eye Sensitivity.

### Test #1

The Soldier is hooked up to EEG diagnostic equipment and sits in front of a PC
With the Webcam is produced the lowest level of Visual Stimulation using the Webcam Level #5
Immediately the level of Visual Stimulation is increased to Level #1 for one minute
The Visual Energy level is lowered to Level #5
The Visual Energy level is increased to Level #1 for one minute
The EEG detects abnormal sensitivity to Visual Stimulation on the highest level

### Test #2

The soldier is hooked up to EEG diagnostic equipment and sits in front of PC
Level Visual Energy #2 is applied on the computer screen for one minute
The Visual Energy level is increased to Level #5 for one minute
If there is a change in readings on the EEG it is an indication that there is some mental underlying problem which may become aggravated in combat. Normal people show no difference in sensitivity between Level #4 and Level #5.

### Treatment of Mental Disorders Using Eye Sensitivity

Diagnosis showing eye contact sensitivity on the Level #1 indicates abnormal stress and the patient needs treatment
Releasing the stress and sensitivity to eye contact through changing the levels of eye contact from Level #1 to #3 to #2 to #3.

Repeat this process eight times or according to the judgment of the therapist administering the treatment.
Go from Level #3 to level #2 several times
Rest at #2
Repeat Diagnostic technique at Level #1

When the sensitivity to eye contact has been removed, the treatment is complete. A period of relaxation with music and narration using eye contact Level #5 is still needed.

It is understood that the preceding description is given merely by way of illustration and not in limitation of the invention and that various modifications may be made thereto without departing form the spirit of the invention as claimed.

What is claimed is:

1. A Visual Stimulus method of analysis using Eye Contact and Eye Movements for diagnosis and treatment of psychological problems, the method comprising:
carrying out a six step Visual Stimulus method of analysis using Eye Contact and Eye Movements for diagnosis and treatment of psychological problems, comprising the six steps of:
looking straight into the eyes of a person;
looking straight at the person face to face as when one person greets the other and they shake hands and smile at each other;
looking straight at the face of the person when the person is looking up;
looking at the face of the person when the person's face is turned to the side;
looking at the person when the person's back is turned;
following the person's finger as it moves slowly in front of the eyes in a procedure called Eye Movement Desensitization and Reprocessing (EMDR).

2. The method of claim 1 wherein a person employs the six steps of the method while looking into a mirror.

3. The method of claim 1 wherein a therapist employs the six steps of the method with a patient.

4. The method of claim 1 wherein a person employs the six steps of the method by looking into a computer screen having the person's image on the screen from a video camera pointed at the person.

5. The method of claim 1 further comprising a software program played on a computer wherein the software program carries out a series of therapeutic steps for a person using the computer, the therapeutic steps providing the therapeutic effects of the six step method.

6. The method of claim 1 further comprising a step of a patient using mirrored glasses to look into a mirror so that the patient can see multiple images of himself in the mirror to produce the effects of visual stimulation devices.

7. The method of claim 1 applied as a technique of Diagnosis of Mental Disorders in their early stages and for recruitment of soldiers for combat using a Web camera and the specific Visual Stimulus described in this disclosure.

8. The method of claim 1 applied as a technique of treatment combining various levels of Visual Stimulation through simulation of Eye Contact using a Web camera on a Personal Computer (PC).

9. The method of claim 1 applied as a visual simulation using a Web camera on a Personal Computer (PC) using the technique used in Psychology Eye Movement Desensitization and Reprocessing (EMDR).

10. The method of claim 1 wherein the person performs the six steps of the method using items provided in a kit, the kit comprising:
a software program for carrying out a series of therapeutic steps for a person using a computer, the therapeutic steps providing the therapeutic effects of the six step method, the software program administered by a professional therapist for the person;
a software program for a person to use on a computer for carrying out a series of therapeutic steps using a computer, the therapeutic steps providing the therapeutic effects of the six step method.

11. A Visual Stimulus method of analysis using a personal computer and a Web camera simulating Eye Contact and Eye Movements for diagnosis and treatment of psychological problems, the method comprising:
a user looking into a computer screen while a Web camera is projecting a real time image of the user on the computer screen;
the user carrying out the six steps of the Visual Stimulus method of analysis simulating Eye Contact and Eye Movements for diagnosis and treatment of psychological problems, comprising the six steps of:
the user looking straight into the user's own eyes on a computer screen;
the user looking straight at the user's image on the computer screen face to face like when one person greets the other, and they shake hands and smile at each other;
the user looking at the user's own face when the user's face is looking up on the computer screen or the visual effect of looking up;
the user looking at the user's own face when the user's face is turned to the side in the computer screen;
the user looking at the user's own image when the user's back is turned in the computer screen;
the user following a finger on the computer screen as it moves slowly in front of the user's eyes in a procedure called Eye Movement Desensitization and Reprocessing (EMDR).

12. The method of claim 11 further comprising a step of a patient looking into the computer screen with a number of images of the patient visible on the computer screen input by the Web camera to simulate a technique of using mirrored glasses to look into a mirror so that the patient can see multiple images of himself in the mirror to produce the effects of visual stimulation devices.
